(19) United States
(12) Patent Application Publication
(10) Pub. No.: US 2024/0207145 A1
(43) Pub. Date: Jun. 27, 2024

(54) COMPOSITION FOR SANITIZING A SURFACE OF SKIN

(71) Applicant: Kao USA Inc., Cincinnati, OH (US)

(72) Inventors: Prajakta Songade, Mason, OH (US); Adam P. Schrott, Hebron, KY (US)

(21) Appl. No.: 18/085,973

(22) Filed: Dec. 21, 2022

(51) Int. Cl. A61K 8/04; A61K 8/34; A61K 8/36; A61K 8/39; A61K 8/60; A61K 8/894; A61Q 17/00

(52) U.S. Cl. CPC A61K 8/046; A61K 8/34; A61K 8/342; A61K 8/361; A61K 8/39; A61K 8/602; A61K 8/894; A61Q 17/005; A61K 2800/596

(57) ABSTRACT

A composition, for sanitizing a surface of skin, includes a PPG silicone. The PPG silicone is a silicone surfactant in the composition. The PPG silicone is present in the composition at an amount ranging from greater than 1% to 6% by weight of the composition. The composition also includes a solvent and a C₁-C₄ alcohol.

### FIELD OF TECHNOLOGY

Disclosed are compositions for sanitizing a surface of skin, as well as methods of making and using the same.

### BACKGROUND

Sanitizers are commonly used to disinfect surfaces and human skin. In particular, hand sanitizers may be used for disinfecting one's hands after using a restroom or touching surfaces that are exposed to the public. Hand sanitizers typically contain a high content of alcohol for disinfecting.

A subset of hand sanitizers includes foaming hand sanitizers, which are usually released from a dispenser, then rubbed onto one's hands. Such hand sanitizers may be in a “leave-on” form, or may be subsequently washed off with water.

Many foaming hand sanitizers include a silicone and a high content (e.g., 50% or greater by weight of a composition) of an alcohol.

A need exists for a foaming hand sanitizer that has improved foam consistency, quality, texture, and/or smaller bubble size. The instant disclosure seeks to address one or more of the aforementioned needs in the art.

### SUMMARY

Disclosed are compositions for sanitizing a surface, in particular a surface of skin. The disclosed compositions may include a polypropylene glycol (PPG) silicone, as a silicone surfactant, in an amount ranging from greater than 1% to 6% by weight.

The disclosed compositions may (1) sanitize a surface of skin by disrupting microbial cells present on the surface of skin and (2) foam when dispensed through a pump. The disclosed compositions may include at least one non-ionic surfactant. Bound by no particular theory, it is believed that the combination of a PPG silicone and at least one non-ionic surfactant allows for the composition to form a stable, robust foam.

### DETAILED DESCRIPTION

A composition for sanitizing a surface of skin is presented. In certain versions, the composition may include a C₁-C₄ alcohol and a PPG silicone. Furthermore, the composition may include at least one non-ionic surfactant. The disclosed compositions were found to have comparable performance to a commercial standard, as described herein.

### Solvent

In one version, the composition may include a solvent for subsuming the remaining ingredients of the composition. The solvent may be present at an amount ranging from 4% to 35%, or from 8% to 30%, or from 13% to 25% by weight of the composition, or in an amount sufficient to total the weight of the composition to 100% (QS). The amount may also range from 4%, or 8%, or 13% to 25%, or 30%, or 35% by weight of the composition, or an amount sufficient to total the weight of the composition to 100% (QS). In one version, the solvent is water, for instance, deionized water.

C₁-C₄ Alcohol

The composition may include an alcohol having 1-4 carbon atoms (henceforth, a C₁-C₄ alcohol) for disinfecting the surface of skin. Bound by no particular theory, it is believed that the C₁-C₄ alcohol acts as an antimicrobial agent to disrupt microbial cells (e.g., bacteria and viruses) present on the surface of skin. The C₁-C₄ alcohol may be present at an amount ranging from 50% to 90%, or from 60% to 80%, or from 65% to 75% by weight of the composition. The amount may also range from 50%, or 60%, or 65% to 75%, or 80%, or 90% by weight of the composition. In one instance, the C₁-C₄ alcohol may be selected from ethanol (i.e., a C₂ alcohol) or isopropyl alcohol (i.e., a C₃ alcohol).

### Fragrance

In one version, the composition may include a fragrance for providing an aesthetic scent to the composition. The fragrance may be present at an amount ranging from 0.05% to 0.4%, or from 0.1% to 0.3%, or from 0.15% to 0.25% by weight of the composition. The amount may also range from 0.05%, or 0.1%, or 0.15% to 0.25%, or 0.3%, or 0.4% by weight of the composition.

### PPG Silicone

The composition may include a silicone as a silicone surfactant. The silicone may be, for instance, a dimethicone chain having at least one silicone atom attached to a polyether group, such as polyethylene glycol (PEG) or polypropylene glycol (PPG). In one version, the silicone may have a structure of X/Y, with X=PEG groups and Y=PPG groups. For example, the composition may include a PPG silicone as a silicone surfactant, having 0 PEG groups (X=0) and more than 1 PPG groups (Y>1). That is, the silicone may be wholly free of (contains 0%) PEG groups and only has PPG groups. Bound by no particular theory, it is believed that the PPG silicone allows the composition to foam and increases wettability of the composition upon the surface of skin during use. The PPG silicone is believed to allow the composition to foam, especially when dispensed through a pump. In some versions, the PPG silicone may be present at an amount ranging from greater than 1% to 6%, or from 2% to 4.5%, or from 2.5% to 4% by weight of the composition. The amount may also range from greater than 1%, or 2%, or 2.5% to 4%, or 4.5%, or 6% by weight of the composition. The amount may also range from 2% to 6%, or from 2.5% to 5.5%, or from 3% to 4.5%, or from 3.5% to 4% by weight of the composition. The amount may also range from 2%, or 2.5%, or 3%, or 3.5% to 4%, or 4.5%, or 5.5%, or 6% by weight of the composition. In one instance, the PPG silicone may be PPG-12 dimethicone, for example, linear PPG-12 dimethicone, for instance SILSOFT™ 900 Fluid available from Momentive Performance Materials.

### Non-Ionic Surfactants

The composition may include at least one non-ionic surfactant. Examples of non-ionic surfactants are as follows:

Glycolipids may be present at an amount ranging from 0.7% to 6%, or from 1.5% to 4.5%, or from 2.5% to 4% by weight of the composition. The amount may also range from 0.7%, or 1.5%, or 2.5% to 4%, or 4.5%, or 6% by weight of the composition. Bound by no particular theory, it is believed that glycolipids act as a booster for foam formation of the composition. Glycolipids have a (di-) rhamnose and carboxylic acid/carboxylate salt groups as heads and saturated hydrocarbons as tails. An example of glycolipids is that sold under the trade name Rheance® One, available from Evonik.

A poloxamer (i.e., a hydrophilic non-ionic block copolymer surfactant having ethylene oxide and propylene oxide blocks) may be present at an amount ranging from 0.2% to 2%, or from 0.5% to 1.5%, or from 0.7% to 1.3% by weight of the composition. The amount may also range from 0.2%, or 0.5%, or 0.7% to 1.3%, or 1.5%, or 2% by weight of the composition. Non-limiting examples of the poloxamer include poloxamer 407 and poloxamer 188.

In one version, a non-ionic polyoxyethylene ether may be present as a further non-ionic surfactant at an amount ranging from 0.1% to 1%, or from 0.2% to 0.6%, or from 0.3% to 0.5% by weight of the composition. The amount may also range from 0.1%, or 0.2%, or 0.3% to 0.5%, or 0.6%, or 1% by weight of the composition. Bound by no particular theory, it is believed that the polyethylene glycol ether increases bulk viscosity of the composition. Non-limiting examples of the non-ionic polyoxyethylene ether include ceteareth-2, ceteareth-3, ceteareth-4, ceteareth-5, ceteareth-6, ceteareth-7, ceteareth-8, ceteareth-9, ceteareth-10, ceteareth-11, ceteareth-12, ceteareth-13, ceteareth-14, ceteareth-15, ceteareth-16, ceteareth-17, ceteareth-18, ceteareth-20, ceteareth-22, ceteareth-23, ceteareth-24, ceteareth-25, ceteareth-27, ceteareth-28, ceteareth-29, ceteareth-30, ceteareth-33, ceteareth-34, and ceteareth-40.

The composition may include combinations of the above non-ionic surfactants.

### Alkyl Glyceryl Ether

The composition may include an alkyl glyceryl ether, which is believed to boost foam formation of the composition. The alkyl glyceryl ether may be present at an amount ranging from 1% to 8%, or from 2% to 6%, or from 3% to 5% by weight of the composition. In other versions, the amount may also range from 1%, or 2%, or 3% to 5%, or 6%, or 8% by weight of the composition. Non-limiting examples of the alkyl glyceryl ether include ethylhexylglycerin and methylheptylglycerin.

### Saturated Fatty Acids and Fatty Alcohols

The composition may include at least one of: (i) a saturated fatty acid and (ii) a fatty alcohol, which are believed to increase stability and elasticity of foam formation of the composition. Saturated fatty acids and fatty alcohols may also limit bubble formation within the foam, thus allowing for a smaller bubble size. The saturated fatty acid and/or the fatty alcohol may be present at an amount ranging from 0.1% to 1%, or from 0.2% to 0.8%, or from 0.3% to 0.7% by weight of the composition. The amount may also range from 0.1%, or 0.2%, or 0.3% to 0.7%, or 0.8%, or 1% by weight of the composition. Non-limiting examples of saturated fatty acids include lauric acid, myristic acid, oleic acid, palmitic acid, and stearic acid. Non-limiting examples of fatty alcohols include cetearyl alcohol, cetyl alcohol, isostearyl alcohol, and stearyl alcohol.

### EXAMPLE COMPOSITIONS

The following are non-limiting examples of the composition as disclosed herein:

Example 1: water (QS), ethanol (80%), PPG-12 dimethicone (2%).

Example 2: water (QS), ethanol (60%), PPG-12 dimethicone (2%).

Example 3: water (QS), ethanol (50%), PPG-12 dimethicone (6%).

Example 4: water (QS), ethanol (60%), PPG-12 dimethicone (6%).

Example 5: water (QS), ethanol (90%), PPG-12 dimethicone (2%), glycolipids (4%).

Example 6: water (QS), ethanol (50%), PPG-12 dimethicone (4%), glycolipids (6%).

Example 7: water (QS), ethanol (70%), PPG-12 dimethicone (6%), glycolipids (6%).

Example 8: water (QS), ethanol (50%), PPG-12 dimethicone (2%), glycolipids (1%).

Example 9: water (QS), ethanol (80%), PPG-12 dimethicone (4%), poloxamer (1%).

Example 10: water (QS), ethanol (65%), PPG-12 dimethicone (3%), poloxamer (0.5%).

Example 11: water (QS), ethanol (85%), PPG-12 dimethicone (5%), poloxamer (2%).

Example 12: water (QS), ethanol (50%), PPG-12 dimethicone (2%), glycolipids (6%), poloxamer (2%).

Example 13: water (QS), ethanol (60%), PPG-12 dimethicone (6%), glycolipids (1%), poloxamer (1%).

Example 14: water (QS), ethanol (75%), PPG-12 dimethicone (4%), glycolipids (6%), poloxamer (0.5%).

Example 15: water (QS), ethanol (70%), PPG-12 dimethicone (5%), glycolipids (5%), poloxamer (1%), non-ionic polyoxyethylene ether (0.5%).

Example 16: water (QS), ethanol (50%), PPG-12 dimethicone (2%), glycolipids (1%), poloxamer (2%), non-ionic polyoxyethylene ether (2%).

Example 17: water (QS), ethanol (65%), PPG-12 dimethicone (3%), glycolipids (3%), poloxamer (0.25%), non-ionic polyoxyethylene ether (0.2%).

Example 18: water (QS), ethanol (55%), PPG-12 dimethicone (6%), glycolipids (1%), poloxamer (0.5%), non-ionic polyoxyethylene ether (0.3%).

Example 19: water (QS), ethanol (85%), PPG-12 dimethicone (6%), glycolipids (1%), poloxamer (0.5%), non-ionic polyoxyethylene ether (0.3%).

Example 20: water (QS), ethanol (65%), PPG-12 dimethicone (4%), glycolipids (1%), poloxamer (2%), non-ionic polyoxyethylene ether (1%), alkyl glyceryl ether (2%).

Example 21: water (QS), ethanol (75%), PPG-12 dimethicone (5%), glycolipids (4%), poloxamer (1%), non-ionic polyoxyethylene ether (0.1%), alkyl glyceryl ether (7%).

Example 22: water (QS), ethanol (65%), PPG-12 dimethicone (4%), glycolipids (1%), poloxamer (2%), non-ionic polyoxyethylene ether (1%), alkyl glyceryl ether (5%).

Example 23: water (QS), ethanol (55%), PPG-12 dimethicone (5%), glycolipids (5%), poloxamer (1%), non-ionic polyoxyethylene ether (0.5%), alkyl glyceryl ether (3%), saturated fatty acid (0.5%).

Example 24: water (QS), ethanol (80%), PPG-12 dimethicone (3%), glycolipids (4%), poloxamer (0.5%), non-ionic polyoxyethylene ether (0.7%), alkyl glyceryl ether (7%), saturated fatty acid (0.2%).

Example 25: water (QS), ethanol (60%), PPG-12 dimethicone (4%), glycolipids (3%), poloxamer (2%), non-ionic polyoxyethylene ether (0.3%), alkyl glyceryl ether (2%), saturated fatty acid (0.7%).

Example 26: water (QS), ethanol (75%), PPG-12 dimethicone (5%), glycolipids (2%), poloxamer (0.3%), non-ionic polyoxyethylene ether (0.9%), alkyl glyceryl ether (4%), saturated fatty acid (0.9%), fatty alcohol (0.2%).

Example 27: water (QS), ethanol (65%), PPG-12 dimethicone (6%), glycolipids (3%), poloxamer (1.5%), non-ionic polyoxyethylene ether (1%), alkyl glyceryl ether (6%), saturated fatty acid (0.6%), fatty alcohol (0.6%).

Example 28: water (QS), ethanol (75%), PPG-12 dimethicone (5%), glycolipids (2%), poloxamer (2%), non-ionic polyoxyethylene ether (0.2%), alkyl glyceryl ether (3%), saturated fatty acid (0.4%), fatty alcohol (0.9%).

### Dynamic Foam Analysis

Dynamic Foam Analysis (DFA) was performed on several test formulations (TF), indicated in Table 1 below (ingredient amounts listed as percent by weight). DFA was also performed on a commercial standard that had ethanol and PEG-12 dimethicone (not PPG-12 dimethicone).

DFA was performed using a KRUSS Dynamic Foam Analyzer—DFA100 with the ADVANCE software for the Dynamic Foam Analyzer—DFA100. Foam Measurement Method of the software was used (Scientific Foam Analysis), with parameters determined for each sample. A gas flow method was utilized, with an internal air source to pump air into a foaming cylinder at a fixed rate. Air flow disrupts a liquid sample in the foaming cylinder to create foam, which the instrument and software then analyze.

TABLE 1
Test Formulations

| Material | TF 1 | TF 2 | TF 3 | TF 4 | TF 5 |
|---|---|---|---|---|---|
| Water | QS to | QS to | QS to | QS to | QS to |
| | 100 | 100 | 100 | 100 | 100 |
| Non-ionic | 0 | 0 | 0 | 0 | 0.3-0.7 |
| Polyoxyethylene | | | | | |
| Ether | | | | | |
| Glycolipids | 0 | 1-6 | 1-6 | 1-6 | 1-6 |
| Alkyl Glyceryl | 0 | 0 | 1-4 | 1-4 | 1-4 |
| Ether | | | | | |
| Saturated Fatty Acid | 0 | 0 | 0 | 0.2-0.75 | 0.2-0.75 |
| Fatty Alcohol | 0 | 0 | 0 | 0.2-0.75 | 0.2-0.75 |
| Ethanol | 60-75 | 60-75 | 60-75 | 60-75 | 60-75 |
| Poloxamer | 0 | 0 | 0.5-1 | 0.5-1 | 0.5-1 |
| PPG-12 | greater | greater | greater | greater | greater |
| Dimethicone | than 1 | than 1 | than 1 | than 1 | than 1 |
| | to 6 | to 6 | to 6 | to 6 | to 6 |

It was found that the above test formulations within the ranges set forth above have comparable DFA performance to the commercial standard.

Percent ranges and point values shown herein are understood to have a +/−1% tolerance.

### Mesh Combinations for Foaming

Table 2 below indicates combinations of top mesh and bottom (lower) mesh sizes that can be used to foam the composition. In one aspect, a composition as disclosed herein may be provided in a container having a top mesh and a bottom mesh combination as provided below.

TABLE 2
Mesh Combinations for Foaming
Mesh Combinations for Foaming

| TOP MESH | BOTTOM (LOWER) MESH |
|---|---|
| 80 | 70 |
| 100 | 60 |
| 100 | 80 |
| 100 | 100 |
| 150 | 100 |
| 200 | 100 |
| 250 | 100 |
| 250 | 150 |
| 255 | 150 |
| 300 | 100 |
| 300 | 150 |
| 300 | 200 |
| 330 | 330 |
| 350 | 100 |
| 350 | 150 |
| 350 | 200 |
| 380 | 150 |

### Air to Liquid Ratios

Air to liquid ratio refers to a volume of an air tank to a volume of a liquid chamber in a pump. The following are non-limiting examples of ratios between air to liquid (air:liquid) that can be used to foam the composition: 7:1, 7.73:1, 9:1, 11:1, 11.19:1, 12:1, 12.59:1, 13:1, 15:1, and 20:1. In one aspect, the composition may be provided in a container having an air to liquid ratio as disclosed above.

### Method of Making

The following is a general, non-limiting method of making the composition:

A first premixture is formed by homogeneously mixing hydrophobic ingredients with alcohol. A second premixture is formed by homogeneously mixing hydrophilic ingredients with deionized water. The second premixture is added to the first premixture to form a pre-final mixture. This is done “cold,” i.e., at room temperature, and homogeneous mixing ensured. Then, all remaining ingredients are added to the pre-final mixture to form a final composition.

### Method of Use

The following are general, non-limiting methods of use of the composition as disclosed herein:

In one aspect, a method of sanitizing a surface (for instance, a surface of skin) may include contacting a composition as disclosed herein with the surface. A composition as disclosed herein may be dispensed from a container having a pump. The pump includes a mesh for foaming the composition.

In another aspect, 0.1 g to 2 g of a composition as disclosed herein is dispensed from a container, having a pump including a mesh, onto a hand of a user. The container may be a free-standing bottle or an upside-down dispenser attached to a wall. The composition may dispensed onto the hand of the user. The user then rubs the amount of the composition into the hand (or a pair of hands) until the amount of the composition is no longer visible, or the hand feels dry. Alternatively, the user may rub the amount of the composition onto any surface of skin (e.g., arms and legs). The surface of skin may be the user's own, or someone else's.

The methods may further comprise administering a composition as disclosed herein, wherein the composition is foamed prior to contact with the surface of skin, and the foaming is carried out by a container having a top mesh and a bottom mesh combination and/or an air to liquid combination as provided herein

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Percent ranges of amounts, and other numerical values, as disclosed herein, are understood to include the recited value+/−1% unless otherwise specified.

All references, including patent applications, patent publications, and non-patent literature, that are referred to in the present specification are incorporated by reference herein, unless it is expressly indicated that they are not incorporated by reference herein.

Having shown and described various aspects of the present invention, further adaptations of the compositions and methods described herein may be accomplished by appropriate modifications by one of ordinary skill in the art without departing from the scope of the present invention. Several of such potential modifications have been mentioned, and others will be apparent to those skilled in the art. For instance, the examples, materials, ratios, steps, and the like discussed above are illustrative and are not required. Accordingly, the scope of the present invention should be considered in terms of any claims that may be presented and is understood not to be limited to the details of composition and operation described in the specification.

What is claimed is:

1. A composition, for sanitizing a surface of skin, comprising:
a solvent;
a C₁-C₄ alcohol; and
from greater than 1% to 6% of a PPG silicone, by weight of said composition.

2. Said composition of claim 1, wherein said PPG silicone is PPG-12 dimethicone.

3. Said composition of claim 1, further comprising at least one non-ionic surfactant.

4. Said composition of claim 3, wherein said at least one non-ionic surfactant is selected from glycolipids, a poloxamer, and combinations thereof.

5. Said composition of claim 1, further comprising an alkyl glyceryl ether.

6. Said composition of claim 1, further comprising a non-ionic polyoxyethylene ether.

7. Said composition of claim 1, further comprising a saturated fatty acid.

8. Said composition of claim 1, further comprising a fatty alcohol.

9. Said composition of claim 1, wherein said solvent is water.

10. Said composition of claim 1, wherein said C₁-C₄ alcohol is ethanol.

11. A composition, for sanitizing a surface of skin, said composition comprising:
water;
ethanol;
from greater than 1% to 6% of PPG-12 dimethicone, by weight of said composition; and
at least one non-ionic surfactant.

12. Said composition of claim 11, wherein said at least one non-ionic surfactant is selected from: glycolipids, a poloxamer, and combinations thereof.

13. Said composition of claim 11, further comprising an alkyl glyceryl ether.

14. Said composition of claim 11, further comprising a non-ionic polyoxyethylene ether.

15. Said composition of claim 11, further comprising a saturated fatty acid.

16. Said composition of claim 11, further comprising a fatty alcohol.

17. Said composition of claim 11, further comprising both a saturated fatty acid and a fatty alcohol.

18. A composition, for sanitizing a surface of skin, said composition comprising:
water;
ethanol;
from greater than 1% to 6% of PPG-12 dimethicone, by weight of said composition;
at least one surfactant comprising:
glycolipids and a poloxamer, and a non-ionic polyoxyethylene ether an alkyl glyceryl ether.

19. Said composition of claim 18, further comprising a non-ionic polyoxyethylene ether and an alkyl glyceryl ether.

20. Said composition of claim 18, further comprising at least one of: (i) a saturated fatty acid and (ii) a fatty alcohol.
